(19) United States
(12) Patent Application Publication
(10) Pub. No.: US 2025/0354132 A1
(43) Pub. Date: Nov. 20, 2025

(54) XYLANASES WITH ENHANCED THERMOTOLERANCE AND USES THEREOF

(71) Applicant: ARCHER-DANIELS-MIDLAND COMPANY, Decatur, IL (US)

(72) Inventors: Aubrey Jones, Davis, CA (US); Kazuhiko Ishikawa; Romil Benyamino, Davis, CA (US)

(21) Appl. No.: 18/861,143

(22) PCT Filed: Apr. 28, 2023
(86) PCT No.: PCT/US2023/066345

§ 371 Date: Oct. 28, 2024

Related U.S. Application Data
(60) Provisional application No. 63/335,925, filed on Apr. 28, 2022.

(51) Int. Cl. C12N 9/24

(52) U.S. Cl. CPC C12N 9/2402; C12Y 302/01008

(57) ABSTRACT

A thermotolerant xylanase enzyme that is a variant of SEQ ID NO:2, wherein the variant comprises at least one amino acid substitution at a position selected from the group consisting of 90, 105, 114, and 115 and/or comprises any of the following sets of substitutions:

(a) G48C and T206C;

(b) Y123V, S125C and N171C;

(c) S114C, Y123V, S125C and N171C;

(d) Y123V, S125C, N171C, G48C and T206C;

(e) S114C, Y123V, S125C, N171C, G48C and T206C;

wherein the substitution or substitutions increases the thermotolerance of the xylanase relative to a parent xylanase lacking the substitution(s)

### TECHNICAL FIELD

The present disclosure relates generally to a thermotolerant xylanase and enzyme-assisted methods using the thermotolerant xylanase.

### BACKGROUND

Xylanase is an important enzyme involved in the breakdown of xylan, a non-starch polysaccharide (NSP) component of plant cell walls. Xylanase enzymes are produced by a variety of bacteria, protozoa, fungi, algae, insects, terrestrial plants and germinating seeds. These enzymes have numerous industrial applications such as clarification of wine and juice, enhanced starch separation, baking, animal feed, and bleaching of paper pulp. Some of these industrial applications involve elevated temperatures that inactivate most enzymes. Consequently, there is a need for new xylanases that have greater thermotolerance than those normally found in nature.

The inclusion of non-starch polysaccharide degrading enzymes (NSPase) such as xylanase in the diets of non-ruminant animals have been shown to have beneficial effects on their health through improved gut physiology. For example, the inclusion of NSPase in poultry feed has been shown to improve the feed digestibility of broilers. The supplementation of enzyme components in poultry feed often involves a high temperature process, sometimes referred to as pelleting. This process, which can involve temperatures reaching 95° C., often has negative effects on the stability of these NSPase components leading to a decrease or total loss of activity of one or more enzyme components. There is a critical need for developing NSPase components that survive high temperature treatments such as pelleting. Addition of NSP-degrading enzymes has also been shown to enhance the separation of starch during wet milling processes by releasing starch, protein and water ensnared in corn fiber that are recalcitrant to mechanical disruption alone. Xylanases that withstand elevated temperatures of the wet milling process are advantageous in giving better separation and recovery of starch and increased yields of starch-derived sugars.

### SEQUENCE LISTING

The instant application contains a Sequence Listing which has been submitted electronically in XML format and is hereby incorporated by reference in its entirety. Said XML copy, created on Apr. 28, 2023, is named “ALL.0046sequencelisting_ST26.xml” and is 4 KB in size.

### SUMMARY

Disclosed herein are enzymes that have greater thermotolerance than conventional enzymes. In an aspect, this disclosure provides xylanases that comprise the amino acid sequence SEQ ID NO:2 but for at least one amino acid substitution at position 90, 105, 114, and/or 115; and/or any of sets of substitutions:
G48C and T206C;
Y123V, S125C, and N171C;
S114C, Y123V, S125C, and N171C;
Y123V, S125C, N171C, G48C, and T206C; or
S114C, Y123V, S125C, N171C, G48C, and T206C;
   wherein the substitution or substitutions increases the thermotolerance of the xylanase relative to a xylanase comprising the amino acid sequence SEQ ID NO:2.

In another aspect, this disclosure provides processes for using the thermotolerant xylanase.

### BRIEF DESCRIPTION OF THE DRAWINGS

FIG. 1 shows the results of a xylanase AZCL-arabinoxylan temperature challenge assay on the supernatants from Saccharomyces cerevisiae expressed Thielavia terrestris GH11 xylanase variants V22, AJ039, AJ040, AJ045 (V45), AJ102, AJ131 (V131) showing improved residual activity relative to wild-type (wt) shown as activity relative to the same sample not treated with heat.

FIG. 2 shows the results of xylanase AZCL-arabinoxylan temperature challenge assay on the supernatants from Trichoderma reesei heterologously expressing Thielavia terrestris GH11 xylanase variants AJ045 (V45) and AJ131 (V131) showing improved residual activity relative to wild-type (wt)) shown as activity relative to the same sample not treated with heat

FIG. 3 shows Cp (KJ/mol/K) versus temperature for variant V45 for first, second, and third melts.

FIG. 4 shows Cp (KJ/mol/K) versus temperature for variant V131 for first, second, and third melts.

### DETAILED DESCRIPTION

A common component in NSPase feed enzyme products is the xylan hydrolyzing activity endo-β-1,4-xylanase (EC 3.2.1.8). A xylanase (SEQ ID NO:2; glycoside hydrolase family GH11) from the thermophilic fungus Thielavia terrestris (Tithe) has been identified and characterized as a lead component in the thermotolerant NSPase disclosed herein. Disclosed herein is a heterologously expressed Thite GH11 xylanase in Trichoderma reesei and Saccharomyces cerevisiae. This xylanase has a melting temperature, determined by Differential Scanning calorimetry (DSC), of approximately 73° C., which potentially renders it inactive in the pelleting process. Using rational site-directed mutagenesis and combinatorial protein engineering, identified herein are amino acid substitutions that statistically improve the residual activity of this xylanase following thermal challenge.

Industrial applications benefiting from thermotolerant xylanases disclosed herein include, but are not limited to animal feed manufacturing, baking, pulp bleaching, fabric bleaching, and converting biomass to biofuel (e.g., ethanol).

In some embodiments, thermotolerant xylanases comprise the amino acid sequence SEQ ID NO:2 but for at least one amino acid substitution at position 90, 105, 114 and/or 115.

In some embodiments, thermotolerant xylanases comprise the amino acid sequence SEQ ID NO:2 but for at one of the following sets of substitutions:
G48C and T206C;
Y123V, S125C, and N171C;
S114C, Y123V, S125C, and N171C;
Y123V, S125C, N171C, G48C, and T206C; and
S114C, Y123V, S125C, N171C, G48C, and T206C.

In some embodiments, thermotolerant xylanases comprise the amino acid sequence SEQ ID NO:2 but for:
a. at least one amino acid substitution at position 90, 105, 114 and/or 115; and/or
b. one of the following sets of substitutions:
   G48C and T206C;
   Y123V, S125C, and N171C;
   S114C, Y123V, S125C, and N171C;
   Y123V, S125C, N171C, G48C, and T206C; and
   S114C, Y123V, S125C, N171C, G48C, and T206C.

In some embodiments, a thermotolerant xylanase comprises the amino acid sequence SEQ ID NO:2 but for at least one substitution selected from the group consisting of S90T, Q105V, Q105I, S114C, S114P, and A115S.

In some embodiments, a thermotolerant xylanase comprises the amino acid sequence SEQ ID NO:2 but for the substitution S90T. In some embodiments, a thermotolerant xylanase comprises the amino acid sequence SEQ ID NO:2 but for the substitution Q105V. In some embodiments, a thermotolerant xylanase comprises the amino acid sequence SEQ ID NO:2 but for the substitution Q105I. In some embodiments, a thermotolerant xylanase comprises the amino acid sequence SEQ ID NO:2 but for the substitution S114C. In some embodiments, a thermotolerant xylanase comprises the amino acid sequence SEQ ID NO:2 but for the substitution S114P. In some embodiments, a thermotolerant xylanase comprises the amino acid sequence SEQ ID NO:2 but for the substitution A115S.

In some embodiments, a thermotolerant xylanase comprises the amino acid sequence SEQ ID NO:2 but for the substitutions S90T and Q105V. In some embodiments, a thermotolerant xylanase comprises the amino acid sequence SEQ ID NO:2 but for the substitutions S90T and Q105I. In some embodiments, a thermotolerant xylanase comprises the amino acid sequence SEQ ID NO:2 but for the substitutions S90T and S114C. In some embodiments, a thermotolerant xylanase comprises the amino acid sequence SEQ ID NO:2 but for the substitutions S90T and S114P. In some embodiments, a thermotolerant xylanase comprises the amino acid sequence SEQ ID NO:2 but for the substitutions S90T and A115S.

In some embodiments, a thermotolerant xylanase comprises the amino acid sequence SEQ ID NO:2 but for the substitutions Q105V and S114C. In some embodiments, a thermotolerant xylanase comprises the amino acid sequence SEQ ID NO:2 but for the substitutions Q105V and S114P. In some embodiments, a thermotolerant xylanase comprises the amino acid sequence SEQ ID NO:2 but for the substitutions Q105V and A115S.

In some embodiments, a thermotolerant xylanase comprises the amino acid sequence SEQ ID NO:2 but for the substitutions Q105I and S114C. In some embodiments, a thermotolerant xylanase comprises the amino acid sequence SEQ ID NO:2 but for the substitutions Q105I and S114P. In some embodiments, a thermotolerant xylanase comprises the amino acid sequence SEQ ID NO:2 but for the substitutions Q105I and A115S.

In some embodiments, a thermotolerant xylanase comprises the amino acid sequence SEQ ID NO:2 but for the substitutions S114C and A115S. In some embodiments, a thermotolerant xylanase comprises the amino acid sequence SEQ ID NO:2 but for the substitutions S114P and A115S.

In some embodiments, a thermotolerant xylanase comprises the amino acid sequence SEQ ID NO:2 but for the substitutions S90T, Q105V, and S114C. In some embodiments, a thermotolerant xylanase comprises the amino acid sequence SEQ ID NO:2 but for the substitutions S90T, Q105V, and S114P. In some embodiments, a thermotolerant xylanase comprises the amino acid sequence SEQ ID NO:2 but for the substitutions S90T, Q105V, and A115S.

In some embodiments, a thermotolerant xylanase comprises the amino acid sequence SEQ ID NO:2 but for the substitutions S90T, Q105I, and S114C. In some embodiments, a thermotolerant xylanase comprises the amino acid sequence SEQ ID NO:2 but for the substitutions S90T, Q105I, and S114P. In some embodiments, a thermotolerant xylanase comprises the amino acid sequence SEQ ID NO:2 but for the substitutions S90T, Q105I, and A115S.

In some embodiments, a thermotolerant xylanase comprises the amino acid sequence SEQ ID NO:2 but for the substitutions Q105V, S114C, and A115S. In some embodiments, a thermotolerant xylanase comprises the amino acid sequence SEQ ID NO:2 but for the substitutions Q105V, S114P, and A115S.

In some embodiments, a thermotolerant xylanase comprises the amino acid sequence SEQ ID NO:2 but for the substitutions Q105I, S114C, and A115S. In some embodiments, a thermotolerant xylanase comprises the amino acid sequence SEQ ID NO:2 but for the substitutions Q105I, S114P, and A115S.

In some embodiments, a thermotolerant xylanase comprises the amino acid sequence SEQ ID NO:2 but for the amino acid substitutions S90T, Q105V, S114C, and A115S.

In some embodiments, a thermotolerant xylanase comprises the amino acid sequence SEQ ID NO:2 but for the amino acid substitutions S90T, Q105V, S114P, and A115S.

In some embodiments, a thermotolerant xylanase comprises the amino acid sequence SEQ ID NO:2 but for the amino acid substitutions S90T, Q105I, S114C, and A115S.

In some embodiments, a thermotolerant xylanase comprises the amino acid sequence SEQ ID NO:2 but for the amino acid substitutions S90T, Q105I, S114P, and A115S.

In some embodiments, a thermotolerant xylanase comprises the amino acid sequence SEQ ID NO:2 but for the substitutions G48C and T206C. In some embodiments, a thermotolerant xylanase comprises the amino acid sequence SEQ ID NO:2 but for the substitutions Y123V, S125C, and N171C. In some embodiments, a thermotolerant xylanase comprises the amino acid sequence SEQ ID NO:2 but for the substitutions S114C, Y123V, S125C, and N171C. In some embodiments, a thermotolerant xylanase comprises the amino acid sequence SEQ ID NO:2 but for the substitutions Y123V, S125C, N171C, G48C, and T206C. In some embodiments, a thermotolerant xylanase comprises the amino acid sequence SEQ ID NO:2 but for the substitutions S114C, Y123V, S125C, N171C, G48C, and T206C.

In some embodiments, a thermotolerant xylanase comprises the amino acid sequence SEQ ID NO:2 but for (a) any of the individual substitutions or combinations of substitutions described in the paragraphs above; and (b) the substitutions G48C and T206C.

In some embodiments, a thermotolerant xylanase comprises the amino acid sequence SEQ ID NO:2 but for (a) any of the individual substitutions or combinations of substitutions described in the paragraphs above; and (b) the substitutions Y123V, S125C, and N171C.

In some embodiments, a thermotolerant xylanase comprises the amino acid sequence SEQ ID NO:2 but for (a) any of the individual substitutions or combinations of substitutions described in the paragraphs above except for S114P; and (b) the substitutions S114C, Y123V, S125C, and N171C.

In some embodiments, a thermotolerant xylanase comprises the amino acid sequence SEQ ID NO:2 but for (a) any of the individual substitutions or combinations of substitutions described in the paragraphs above; and (b) the substitutions Y123V, S125C, N171C, G48C, and T206C.

In some embodiments, a thermotolerant xylanase comprises the amino acid sequence SEQ ID NO:2 but for (a) any of the individual substitutions or combinations of substitutions described in the paragraphs; and (b) the substitutions S114C, Y123V, S125C, N171C, G48C, and T206C.

These amino acid substitutions increase the thermotolerance of the thermotolerant xylanases relative to the Thite xylanase comprising the amino acid sequence SEQ ID NO:2. “Thermotolerance” is defined as an increase in residual enzyme activity relative to the wild-type or parental enzyme following a thermal challenge or step that involves incubating the enzyme at temperatures above 60° C. Increased thermotolerance can be conferred by modifying the amino acid sequence or composition of the molecule such that it denatures at a higher temperature than the wild-type or parental enzyme. Increased thermotolerance can also be imparted by amino acid modifications or substitutions that enable the enzyme to re-fold following denaturation or thermal inactivation.

The term “wild-type” is herein defined as a xylanase having the amino acid sequence SEQ ID NO:2. Also known as the parent or parental enzyme, the wild-type xylanase can be heterologously expressed in naturally occurring microorganisms such as bacteria, yeast and filamentous fungus.

The term “variant” is herein defined as a xylanase in which the amino acid SEQ ID NO:2 has been modified by one or more amino acid substitutions relative to SEQ ID NO:2. Variant enzymes can be heterologously expressed in naturally occurring microorganisms such as bacteria, yeast and filamentous fungus.

“Improved thermostability,” also known as “improved residual activity,” is herein defined as an improved retention in enzyme activity following a defined elevated temperature challenge, relative to wild-type enzyme activity.

“Improved thermotolerance” is herein defined as an improved capacity for structural re-folding and retention in enzymatic activity following thermal denaturation or thermal inactivation, relative to wild-type structural re-folding and wild-type enzyme activity.

Thermotolerant xylanases disclosed herein can be identified using methods well known in the art. Such methods involve substitution of amino acid residues in a polypeptide sequence followed by screening of the resulting variants for increased thermotolerance (see for example: H. Yang et al. 2015. Chem. Bio. Eng. Rev. 2: 87-94; X. F. Zhang et al. 2016. Sci. Rep. 6: 33797; H. Yu et al. 2017. Sci. Rep. 7: 41212; Z. Xu et al. 2020. Crit. Rev. Biotechnol. 40: 83-98).

### Processes Using Thermotolerant Xylanases

The thermotolerant xylanases disclosed herein may be used in various processes. In an aspect, the thermotolerant xylanases disclosed herein may be used in a process selected from the group consisting of wet-milling and dry-grind com milling. The milling process may comprise steeping, i.e., soaking of an organic material in a liquid (usually water) to extract flavors and/or to soften the organic material. Use of the thermotolerant xylanases disclosed herein allows for improved co-product recovery, accelerated steeping kinetics, manufacturing energy savings, and value-additions in the form of enriched mill streams, including, but not limited to, light steepwater/saccharification tank effluent to fermenters. Use of the thermotolerant xylanases disclosed herein can be readily implemented in wet milling processes with minimal alteration to the manufacturing plant.

In an aspect, processes using the thermotolerant xylanases disclosed herein have advantages and provide benefits over processes involving conventional wild-type enzymes. In an aspect, processes disclosed herein include enzyme preparation wherein spray-dried thermotolerant NSPase disclosed herein has a higher activity per unit enzyme protein (>5,000XU/g) than a solid-form conventional wild-type enzyme protein (˜3,500 XU/g).

In an aspect, processes disclosed herein include enzyme preparation wherein spray-dried thermotolerant NSPase disclosed herein has significantly higher activity and thermotolerance compared to conventional liquid-based treatment. As such, lower enzyme dosing of thermotolerant NSPase disclosed herein is needed relative to the steep or mash solids to gain an effect superior to conventional products.

NSPase disclosed herein has a greater demonstrated thermotolerance relative to other treatments, e.g., Frontia® Fiberwash (a product having a wild-type xylanase, offered by Novozymes A/S), and improved performance in real-world steeping and fiber-washing conditions relative to such treatments as well.

In an aspect. processes disclosed herein require no modification to manufacturing plant layout, compared to conventional processes that require, for example, installation of an incubation tank and piping to store first-grind corn during enzyme treatment prior to second-grind. Further, unlike conventional products that must be added after the milling process to the fiber tank, thermotolerant NSPase disclosed herein can be dosed directly into steep water tanks and/or mixed with corn entering the steep and still improve downstream separations, e.g., reducing total starch in fiber.

The above approach runs contrary to the view by some skilled in the art that addition of cellulolytic and xylanolytic enzymes added to the steep serve no purpose, especially when one considers that the steepwater is recirculated back into the steep. Once the corn is screened out, it will carry only a nominal amount of active enzyme protein with it forward into the mill. As such, the bulk of the enzyme activity will be retained on the steeping side, requiring only a maintenance dose once the target activity in the steeps has been achieved. Steeping also requires lower SO₂ concentrations in the presence of thermotolerant NSPase disclosed herein, while releasing additional fermentable C6 and C5 sugars from the grain. From an ethanol fermentation standpoint, a higher sugar concentration in steepwater will require lower energy inputs to achieve a desired dextrose equivalent (DE) target through evaporation, and will have lower concentrations of SO₂, reducing stress on the yeast. On the steeping side, the presence of C5 and C6 sugars and reduced SO₂ serve to improve the health of the steep microbiome—encouraging the growth of native lactobacillus.

The presence of the enzyme within the steepwater has also been shown to improve germ separations, making germ more buoyant in the steepwater mixture, facilitating easier separations of a germ fraction during the wet milling process. Steepwater has higher magnesium, potassium, and phosphorous levels relative to standard milling conditions during the steeping process when enzyme is present in the steepwater. This aspect is important, as steepwater typically is concentrated and combined with corn gluten feed. The processes disclosed herein provides an enriched feed material relative to that produced by conventional processes that do not include use of a xylanase. Elements such as calcium and sodium also are present in higher levels in enzyme treated steepwater. While also beneficial to animal health, these minerals also are known to improve the activity of certain enzymes, e.g., alpha-amylases used in downstream fermentations, as well as that of xylanases common in animal feeds and dosed into the corn steeps themselves. Zinc has also been identified as a promoter of xylanolytic enzyme activity. Manganese and boron levels are also elevated in the processes disclosed herein. Boron improves mineral uptake in livestock, while manganese has been shown to ward off fatty liver disease in cattle. Both elements are also used by metalloenzymes well.

Inositol levels in steepwater are increased in the presence of an enzyme treatment, while phytate/phytic acid levels are lower. From an animal nutrition standpoint, lower levels of phytate equate to lower levels of a metal-ion chelating agent, promoting mineral uptake in livestock. Inositol has been identified as a potential growth promoter in poultry, and an essential micronutrient for aquaculture.

From a commercial operations standpoint, the processes disclosed herein can deliver starch-in-fiber reductions in excess of 20% with a 0.1% (preliminary) dosing of enzyme protein to un-steeped corn on a wild-type basis, a >20% increase over that achieved using conventional products.

A 10% reduction of bound starch-in-fiber in a wet milling unit operation can equate to approximately a >$10 MM/year cost savings and reduce the amount of corn that must be ground to reach production targets.

In addition, the steepwater produced from the steeping process contains 20% more C5 and C6 sugars (combined) relative to baseline conditions. This encourages the growth of Lactobacillus species, but does nothing to create favorable conditions for Acetobacter species (by 24-40 hours, lactic acid concentrations exceed 3000 ppm, while acetic acid remains effectively unchanged).

From an enzyme dosing cost standpoint, using a thermotolerant, solid enzyme is preferential in plant conditions, where internal temperatures and humidity can vary significantly throughout the year. Solid material in supersacks can be stored in a dark, dry place and require no refrigerated tank to ensure that viability/activity is maintained over time. High activity per unit protein is preferential, as a lower dosing regimen is needed. This, coupled with the improved separations delivered in the mill, and reduced energy demands throughout, creates a highly desirable cost-savings for both batch and continuous steeping (the latter of which has been viewed as an inferior process relative to batch steeping). The enzymes disclosed herein are also viable in a liquid form, which can be easier to pump and dose than solid form. The thermotolerant enzymes disclosed herein are desirable in liquid form in instances where tank refrigeration fails or mill heating equipment malfunctions. Effectively, the thermotolerant enzymes disclosed herein remain viable for intended use without loss of an entire tank of enzyme if temperatures spike higher to the 60-70° C. range.

Those skilled the art, having the benefit of the present disclosure, will recognize that the processes disclosed herein provide cleaner separations with lower energy input than conventional processes. Corn processors will recognize that the processes disclosed herein allows enzymatic wet milling to be more economically feasible. Currently, enzymatic wet milling is not widely practiced in the United States.

Aspects and benefits of the present invention are further described in the following examples.

### Example 1

This example describes identification of several amino acid substitutions that improve the thermotolerance of xylanase. A yeast (S. cerevisiae) codon optimized Thite GH11 xylanase coding sequence (SEQ ID NO:1) was synthesized and subcloned into the yeast expression plasmid pYES2 (Invitrogen, Carlsbad, CA) to generate pAJ021. This plasmid was used as a backbone to generate wild-type Thite GH11 xylanase and to design Thite GH11 xylanase enzyme variants. Variants were designed using rational design augmented with protein engineering design software.

Screening for thermostability was facilitated by transforming and expressing wild-type xylanase and variants xylanases in S. cerevisiae. The construction of these variants was accomplished using two different approaches. Single site substitutions were generated for each variant position using yeast gap-repair described by Raymond et al. (Ref: General Method for Plasmid Construction Using Homologous Recombination, BioTechniques 26:134-141 January 1999) and specific left and right PCR fragments. Each PCR fragment was designed to incorporate specific amino acid substitutions on the 3′ and 5′ ends of the left and right fragments, respectively. The 3′ end of the left PCR fragment was designed to contain 37 bp overlapping sequence homology to pYES2 located downstream the Gal1 promoter and the 3′ end of the right PCR fragment was designed to contain 25 bp of overlapping sequence homology to pYES2 located upstream the CYC1 terminator. The left PCR was designed to contain a yeast consensus sequence (CACAAA) immediately upstream the ATG methionine. The left and right PCR fragments were combined with the pYES2 vector that had been restriction digested with HindIII and XbaI and then used in a yeast transformation similar to Dohmen et al. (Ref: An Efficient Transformation Procedure Enabling Long-term Storage of Competent Cells of Various Yeast Genera. Yeast 7: 691-692). Transformants containing the repaired sequences were selected SC-U selective plates. Combined or multiple substitution variants were constructed using synthetic fragments generated from outside synthetic DNA services. These fragments were subcloned into the pYES2 vector and transformed similarly. Colonies from the transformations exhibiting uracil prototrophy were selected and grown in 96-deep well (1.1 ml) containing 500 μl of SC-U medium with 2% glucose at 30°° C. with 1000 RPM on an Infors shaker. After 24 hrs., the culture broth was centrifuged at 700×g and replaced with SC-U plus 2% galactose. Cultures were grown similarly for 72 hrs., and then the sample broth was harvested by centrifugation at 700×g for 10 min. Variant sample broths were screened using a heat challenge AZCL-Arabinoxylan (Megazyme, Bray, Ireland) assay.

### Example 2

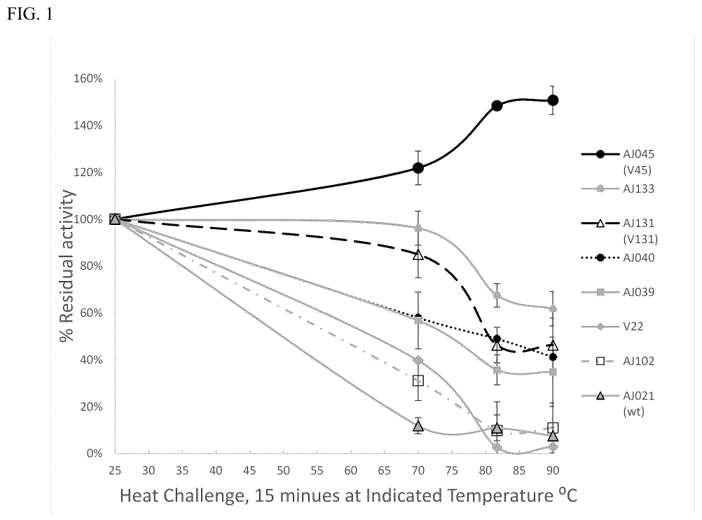

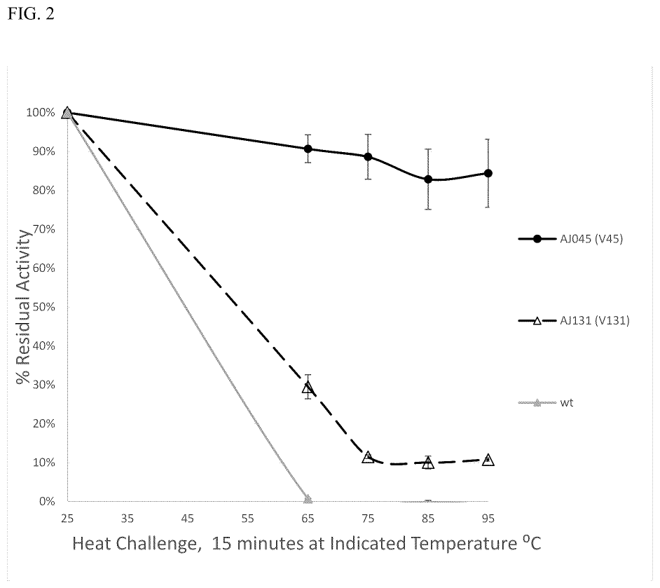

The xylanase activity in culture supernatants was determined using AZCL-arabinoxylan, an insoluble, finely granulated wheat arabinoxylan substrate (Megazyme, Bray. Ireland). The substrate was prepared at a concentration of 0.2% AZCL-arabinoxylan in 50 mM sodium citrate buffer, pH 4.2, and reactions were incubated at 50° C. (FIG. 1). Further enzyme evaluations were performed in reaction conditions at pH 5.3 and incubated at 37° C. (FIG. 2). For thermostability tests, supernatant samples were heat challenged at temperatures for five minutes as indicated in FIG. 1 and FIG. 2. Enzyme activity (hydrolysis of the substrate) was measured by release of AZCL and absorbance detected at 595 nm. Percent residual activity was calculated by comparing activity of enzyme samples treated at elevated temperatures to the same samples not heat challenged.

Eleven variants (incorporating nine amino acid positions) exhibited improved thermotolerance as measured by residual activity when compared to wild-type (wt) Thite GH11 xylanase. See FIG. 1 and Table 2 below. FIG. 1 shows the results of an AZCL-arabinoxylan challenge assay of supernatants from Saccharomyces cerevisiae expressed Thielavia terrestris GH11 xylanase variants V22, AJ039, AJ040, AJ045 (V45), AJ102, AJ131 (V131) showing improved residual activity relative to wild-type. The variants are shown in Table 1 below.

TABLE 1

| Claimed Variants | Amino Acid Substitution(s) |
|---|---|
| Variant 17 | A115S |
| Variant 22 | S114C |
| Variant 24 | S114P |
| Variant 40 | Q105V |
| Variant 42 | Q105I |
| AJ039 | Y123V, S125C, N171C |
| AJ040 | G48C, T206C |
| AJ045 (V45) | Y123V, S125C, N171C, G48C, T206C |
| AJ102 | S90T |
| AJ131 (V131) | S114C, Y123V, S125C, N171C |
| AJ133 | S114C, Y123V, S125C, N171C, G48C, T206C |

Thielavia terrestris GH11 xylanase improved variants and respective designed amino acid substitutions involved in improved xylanase thermostability.
Note:
The amino acid substitution number refers to its position relative to the N-terminal amino acid of the mature protein.

Table 2 shows the results of an AZCL-arabinoxylan challenge assay of supernatants from Saccharomyces cerevisiae heterologously expressing Thielavia terrestris GH11 xylanase, variants with improved residual activity relative to wild-type. Variant V17, V22; V24, V40 ,V42.

TABLE 2

| | Mean % Residual Activity | Standard |
|---|---|---|
| Thielavia terrestris GH11 | of non-heated sample (15 | Deviation |
| xylanase variant # | minutes at 65° C.) | (n of 3) |
| 17 | 19.0% | 2.6% |
| 22 | 59.2% | 7.6% |
| 24 | 18.3% | 4.2% |
| 40 | 19.0% | 1.4% |
| 42 | 28.0% | 4.2% |
| Thielavia terrestris GH11 | 7.6% | 3.2% |
| xylanase wild-type | | |

### Example 3

Expression and thermotolerance of AJ045 (aka V45; Y123V, S125C, N171C, G48C, T206C) and AJ131 (aka V131; S114C, Y123V, S125C, N171C) in Trichoderma reesei. Four V131 and five V45 T. reesei lab scale fermenters were run and culture supernatants were tested in an AZCL-arabinoxylan heat challenge activity assay. Results indicated that the improved residual activity profile observed in variant V45 when expressed in S. cerevisiae is very similar to the improvement seen when this variant is produced in T. reesei.

### Example 4

Differential scanning calorimetry (DSC) was determined for yeast and T. reesei expressed V22, and DSC was determined for yeast expressed AJ045 and AJ131 and is shown in the Table 3 below.

TABLE 3

| Sample (all purified) | Tm |
|---|---|
| Wild-type T. terrestris GH11 from S. cerevisiae (AJ021) | 73.6 |
| Wild-type T. terrestris GH11 from T. reesei | 73.2 |
| V22 from S. cerevisiae | 76.6 |
| AJ045(V45) from S. cerevisiae | 74.5 |
| AJ131 (V131) from S. cerevisiae | 77.7 |

FIG. 2 shows the results of AZCL-arabinoxylan challenge assay of supernatants from Trichoderma reesei heterologously expressing Thielavia terrestris GH11 xylanase variants AJ045 (V45) and AJ131 (V131) showing improved residual activity relative to wild-type. Of particular interest are the thermotolerance characteristics of AJ045. While this variant clearly shows superior performance in an AZCL-arabinoxylan challenge assay (FIG. 1 and FIG. 2), this variant shows little improvement in DSC Tm when compared to wild-type GH11 xylanase. However, the ability of AJ045 to refold after the protein has been subject to a 10-minute melting treatment may be of particular importance in the animal feed industry.

### Example 5

Using Differential Scanning calorimetery (DSC), refolding experiments were performed on purified wild-type Thielavia terrestris GH11 xylanase, V22, V45 and V131 expressed in and purified from Saccharomyces cerevisiae. Samples were purified in 2-steps: 1) captured by self-packed Capto Phenyl ImpRes (Hydrophobic Interaction Chromatography) and 2) polished by HiLoad 16/600 Superdex 75 (Size Exclusion Chromatography). In the DSC, the purified samples were first heated to a few degrees above Tm (10-15 minute ramp time). After melting, the samples were allowed to equilibrate to 25° C. and then they were subject to a typical DSC run at 90° C./hr ramp up to 110° C. Results are shown in Table 4 table below.

TABLE 4
1^{st} and 2^{nd} melt Tm and ΔH for xylanases
expressed in Saccharomyces cerevisiae.

| Purified | 1st Melt | | | |
|---|---|---|---|---|
| Sample | Tm, ° C. | ΔH, kJ/mol | Tm, ° C. | ΔH, kJ/mol |
| wild-type* | 73.2 | 778 | <LOD | <LOD |
| wild-type | 73.6 | 774 | <LOD | <LOD |
| V22 | 76.6 | 732 | <LOD | <LOD |
| V45 | 74.5 | 699 | 74.6 | 330 |
| V131 | 77.7 | 720 | <LOD | <LOD |

*expressed in filamentous fungal host

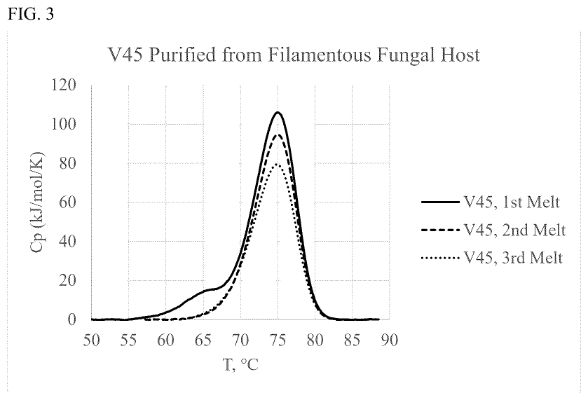

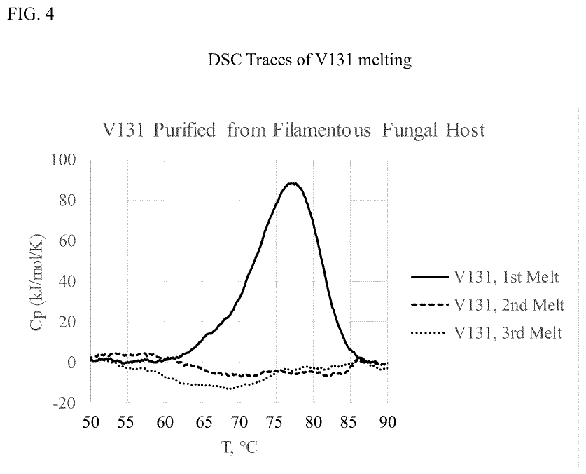

The refolding characteristic of V45 and increased melting temperature of V131 was then verified with expression of the variants in a filamentous fungal host. V45 and V131 were expressed and purified from a filamentous fungal host and tested in a refolding DSC experiment with the addition of a 3^{rd} melt step. The results for V45 and V131 are shown in Tables 5 and 6, respectively; their corresponding DSC thermograms are shown in FIG. 3 and FIG. 4. The DSC thermograms display heat capacity, Cp (KJ/mol/K) versus temperature, T (° C.) for first, second, and third melts. Since protein unfolding is an endothermic process, it is observed as a positive displacement in the thermogram. The midpoint of the transition (peak) is the Tm, and the area under the transition is the enthalpy (ΔH) of the protein's unfolding process.

TABLE 5
V45 Tm and ΔH for each melt

| V45 Melt | Tm, ° C. | ΔH₁ (kJ/mol) |
|---|---|---|
| 1st | 74.6 | 783 |
| 2nd | 74.6 | 673 |
| 3rd | 74.5 | 590 |

TABLE 6
V131 Tm and ΔH for each melt

| V131 Melt | Tm, ° C. | ΔH₁ (kJ/mol) |
|---|---|---|
| 1st | 76.5 | 984 |
| 2nd | <LOD | <LOD |
| 3rd | <LOD | <LOD |

### Example 6

Table 7 shows use of the thermotolerant NSPase Thielavia xylanase variant disclosed herein (referred to as “tNSPase” in Table 6) in comparison to wild-type xylanase in com wet milling. In this example, 400 g dry corn, 466 g deionized water (DI), 0.74 g sodium metabisulfite, and 14.01 g lactic acid were combined in a 1 L bottle. The bottle was placed into a shaking water bath set at 52° C. Corn was steeped for 40 hours. Steepwater was sampled at 0, 16, 24, and 40 hours, with the concentrations of the below compounds measured by HPLC. Phytate, free lactic acid, dextrose, xylose, fructose, inositol, and galactose are desirable in various applications, e.g., ethanol fermentation. Free acetic acid, however, is undesirable in various applications, e.g., ethanol fermentation. The tNSPase of this example is V45 identified above. As shown in Table 7, use of the NSPase of the present invention provides better or similar results to use of wild-type xylanase, even though the tNSPase enzyme loading wt/wt % was less than wild-type xylanase, i.e., 0.06% compared to 0.11%, 0.04% compared to 0.07%, and 0.02% compared to 0.04%.

TABLE 7

| | | | | | |
|---|---|---|---|---|---|
| Enzyme | | | | | |
| protein | | | | | |
| loading | Enzyme | | | | |
| wt/wt % | Used | 0 hrs | 16 hrs | 24 hrs | 40 hrs |

Phytate (g/kg)

| | | | | | |
|---|---|---|---|---|---|
| 0.11% | Wild-type | 163 | 2900 | 4280 | 4020 |
| | Xylanase | | | | |
| 0.06% | tNSPase | 1510 | 3640 | 4410 | 4810 |
| 0.07% | Wild-type | 997 | 3630 | 4100 | 4520 |
| | Xylanase | | | | |
| 0.04% | tNSPase | 1040 | 3340 | 4380 | 4890 |
| 0.04% | Wild-type | 1110 | 3450 | 4000 | 4700 |
| | Xylanase | | | | |
| 0.02% | tNSPase | 1520 | 3700 | 4450 | 4840 |

Free Lactic Acid in Steepwater (g/kg)

| | | | | | |
|---|---|---|---|---|---|
| 0.11% | Wild-type | 6.376 | 6.003 | 5.923 | 5.981 |
| | Xylanase | | | | |
| 0.06% | tNSPase | 6.297 | 5.915 | 5.951 | 5.901 |
| 0.07% | Wild-type | 6.540 | 5.989 | 5.971 | 6.033 |
| | Xylanase | | | | |
| 0.04% | tNSPase | 6.370 | 5.912 | 6.005 | 5.631 |
| 0.04% | Wild-type | 6.401 | 5.976 | 6.009 | 5.906 |
| | Xylanase | | | | |
| 0.02% | tNSPase | 6.448 | 5.961 | 5.966 | 5.793 |

Acetic Acid Formed in Steepwater (g/kg)

| | | | | | |
|---|---|---|---|---|---|
| 0.11% | Wild-type | 0.000 | 0.433 | 0.452 | 0.549 |
| | Xylanase | | | | |
| 0.06% | tNSPase | 0.000 | 0.427 | 0.455 | 0.507 |
| 0.07% | Wild-type | 0.000 | 0.457 | 0.453 | 0.521 |
| | Xylanase | | | | |
| 0.04% | tNSPase | 0.000 | 0.403 | 0.444 | 0.513 |
| 0.04% | Wild-type | 0.000 | 0.407 | 0.443 | 0.509 |
| | Xylanase | | | | |
| 0.02% | tNSPase | 0.000 | 0.401 | 0.475 | 0.522 |

Dextrose in Steepwater (g/kg)

| | | | | | |
|---|---|---|---|---|---|
| 0.11% | Wild-type | 0.116 | 6.508 | 7.893 | 9.06 |
| | Xylanase | | | | |
| 0.06% | tNSPase | 0.506 | 6.470 | 7.949 | 9.17 |
| 0.07% | Wild-type | 0.000 | 6.490 | 7.825 | 9.15 |
| | Xylanase | | | | |
| 0.04% | tNSPase | 0.350 | 6.299 | 7.720 | 8.34 |
| 0.04% | Wild-type | 0.000 | 5.800 | 7.066 | 7.93 |
| | Xylanase | | | | |
| 0.02% | tNSPase | 0.179 | 5.778 | 7.162 | 8.33 |

Xylose in Steepwater (g/kg)

| | | | | | |
|---|---|---|---|---|---|
| 0.11% | Wild-type | 0.000 | 0.000 | 0.000 | 0.000 |
| | Xylanase | | | | |
| 0.06% | tNSPase | 0.000 | 0.000 | 0.000 | 0.131 |
| 0.07% | Wild-type | 0.000 | 0.000 | 0.000 | 0.000 |
| | Xylanase | | | | |
| 0.04% | tNSPase | 0.000 | 0.000 | 0.000 | 0.137 |
| 0.04% | Wild-type | 0.000 | 0.000 | 0.000 | 0.000 |
| | Xylanase | | | | |
| 0.02% | tNSPase | 0.000 | 0.000 | 0.000 | 0.156 |

Fructose in Steepwater (g/kg)

| | | | | | |
|---|---|---|---|---|---|
| 0.11% | Wild-type | 0.000 | 3.968 | 4.676 | 5.479 |
| | Xylanase | | | | |
| 0.06% | tNSPase | 0.000 | 3.756 | 4.728 | 5.268 |
| 0.07% | Wild-type | 0.000 | 3.948 | 4.843 | 5.617 |
| | Xylanase | | | | |
| 0.04% | tNSPase | 0.000 | 3.864 | 4.778 | 5.145 |
| 0.04% | Wild-type | 0.000 | 3.932 | 4.696 | 5.355 |
| | Xylanase | | | | |
| 0.02% | tNSPase | 0.000 | 4.034 | 4.791 | 5.403 |

Inositol in Steepwater (g/kg)

| | | | | | |
|---|---|---|---|---|---|
| 0.11% | Wild-type | 0.000 | 0.205 | 0.187 | 0.207 |
| | Xylanase | | | | |
| 0.06% | tNSPase | 0.000 | 0.128 | 0.209 | 0.199 |
| 0.07% | Wild-type | 0.000 | 0.200 | 0.207 | 0.237 |
| | Xylanase | | | | |
| 0.04% | tNSPase | 0.000 | 0.163 | 0.190 | 0.202 |
| 0.04% | Wild-type | 0.000 | 0.187 | 0.193 | 0.238 |
| | Xylanase | | | | |
| 0.02% | tNSPase | 0.000 | 0.152 | 0.170 | 0.231 |

Galactose in Steepwater (g/kg)

| | | | | | |
|---|---|---|---|---|---|
| 0.11% | Wild-type | 0.000 | 0.258 | 0.387 | 0.604 |
| | Xylanase | | | | |
| 0.06% | tNSPase | 0.000 | 0.158 | 0.274 | 0.664 |
| 0.07% | Wild-type | 0.000 | 0.286 | 0.420 | 0.772 |
| | Xylanase | | | | |
| 0.04% | tNSPase | 0.000 | 0.168 | 0.250 | 0.633 |
| 0.04% | Wild-type | 0.000 | 0.272 | 0.330 | 0.402 |
| | Xylanase | | | | |
| 0.02% | tNSPase | 0.000 | 0.205 | 0.408 | 0.707 |

The processes and use of the thermotolerant xylanase disclosed herein can provide advantages over processes that use conventional wild-type enzymes. These advantages include the following:
Reducing the SO₂ chemical addition during the wet milling process to treat com kernels; without being bound by theory, it is believed that the thermotolerant xylanase of the present disclosure assists and/or promotes chemical penetration and reactions with corn kernels to a greater extent than conventional wild-type enzymes. Compared to a traditional dosage of 2000 ppm SO₂ when using conventional wild-type enzymes, it was found that as little as 500 ppm was necessary in the presence of the thermotolerant xylanase disclosed herein to comparably soften corn kernels.
The thermotolerant xylanase of the present invention has higher activity than conventional wild-type enzymes. Thus, the thermotolerant xylanase of the present invention can be used in lower dosage than conventional wild-type enzymes. See Table 6 above.
The thermotolerant xylanase has higher residual enzyme activity relative to the wild-type or parental enzyme following a thermal challenge or step that involves incubating the enzyme at temperatures above 60° C.

Those skilled in the art, having the benefit of the present disclosure, will recognized that the thermotolerant Thielavia xylanase variant of the present disclosure can be used at low dosing levels to liberate (hemi) cellulose-type material in the form of C5 and C6 saccharides from dry-grind com. The additional benefit gained would be higher ethanol yields.

Those skilled in the art, having the benefit of the present disclosure, will recognized that the thermotolerant Thielavia xylanase variant of the present disclosure can be used in continuous steeping apparatus and methods.

Those having skill in the art, with the knowledge gained from the present disclosure, will recognize that various changes can be made to the disclosed compositions and processes in attaining the advantages disclosed herein and other advantages, without departing from the scope of the present disclosure. As such, it should be understood that the features of the disclosure are susceptible to modifications and/or substitutions. The specific embodiments illustrated and described herein are for illustrative purposes only, and not limiting of the invention as set forth in the appended claims.

What is claimed is:

1. A first xylanase comprising the amino acid sequence SEQ ID NO:2 but for one amino acid substitution at a position selected from the group consisting of 90, 105, 114, and 115 of SEQ ID NO:2 and/or any of sets of substitutions selected from the group consisting of:
(a) G48C and T206C;
(b) Y123V, S125C, and N171C;
(c) S114C, Y123V, S125C, and N171C;
(d) Y123V, S125C, N171C, G48C, and T206C; or
(e) S114C, Y123V, S125C, N171C, G48C, and T206C;
wherein the amino acid substitution or substitutions increases thermotolerance of the xylanase relative a second xylanase comprising the amino acid sequence SEQ ID NO:2.

2. The first xylanase of claim 1 wherein the at least one substitution is selected from the group consisting of S90T, Q105V, Q105I, S114C, S114P, and A115S.

3. The first xylanase of claim 1, wherein the amino acid substitution comprises Y123V, S125C, N171C, G48C, and T206C.

4. A process using the first xylanase of claim 1, wherein the process is a milling process.

5. The process of claim 4, wherein the milling process is a corn milling process.

6. The process of claim 5, wherein the corn milling process is selected from the group consisting of wet-milling and dry-grind milling.

7. A process using the first xylanase of claim 3, wherein the process is a milling process.

8. The process of claim 7, wherein the milling process is a corn milling process.

9. The process of claim 8, wherein the corn milling process is selected from the group consisting of wet-milling and dry-grind milling.

10. The process of claim 4, wherein the process comprises pelleting.

11. The process of claim 10, wherein the pelleting comprises forming pellets comprising the first xylanase and an animal feed.

12. An animal feed comprising the first xylanase of claim 1.

13. The animal feed of claim 12, wherein the animal feed is selected from the group consisting of a poultry feed and an aquaculture feed.

14. A process using the first xylanase of claim 1, wherein the process comprises a thermal challenge that involves subjecting the first xylanase to a temperature above 60° C.

15. A process using the first xylanase of claim 1, wherein the process is selected from the group consisting of animal feed manufacturing, baking, pulp bleaching, fabric bleaching, and converting biomass to biofuel.

16. The process of claim 15, wherein the process is converting biomass to biofuel.

17. The process of claim 16, wherein the biofuel comprises ethanol.


SEQUENCE LISTING (2 sequences)

SEQ ID NO: 1 - DNA, 687 bp, synthetic construct
  misc_feature 1..687: note = codon-optimized coding sequence for SEQ ID NO:2
  source 1..687: mol_type = other DNA
atggtcagttttacctacttggccttgttggccagtagtgttcttggtgccgttgccgctccagctgctgaaccagctggagccgacttgccagatttcgagtttaacatcgaggctagaaaccttactagaaggcaagattacaaccaaaactggaagggtggtggaggttctgtccagtactctgctaccagtaacggttactctgtcaccttctctggagccggtgacttcgtcgttggaaaaggttggaggaccggtagtgcctctaggaccatcaagtaccaaggtaacacccaagcccaagctggtaccgtccttttgtctgtctacggttggtctaggaacccattggtcgagtactacgtccagcagtacacctctaacggtcaaggttctgcccaaggtcagaaattgggtacctacgagagtgacggtgccacctatgagatctggaagcaccaacaagttaaccagccatctatcgagggtaccagtaccttctggcagtacatctctaatagagtctctggttctgccccaacttctggtactgtcaccatggccaaccacatcaatgcttggaaacagcacggattgaacttgggtcagcttgactaccaagttttggccaccgaaggttggggtggtgcctctggaagttcttcttacaccatttctggttaa

SEQ ID NO: 2 - protein, 228 aa, Thielavia terrestris
MVSFTYLALLASSVLGAVAAPAAEPAGADLPDFEFNIEARNLTRRQDYNQNWKGGGGSVQYSATSNGYSVTFSGAGDFVVGKGWRTGSASRTIKYQGNTQAQAGTVLLSVYGWSRNPLVEYYVQQYTSNGQGSAQGQKLGTYESDGATYEIWKHQQVNQPSIEGTSTFWQYISNRVSGSAPTSGTVTMANHINAWKQHGLNLGQLDYQVLATEGWGGASGSSSYTISG